(19) United States
(12) Patent Application Publication
(10) Pub. No.: US 2024/0268328 A1
(43) Pub. Date: Aug. 15, 2024

(54) GENETICALLY MODIFIED POTATO WITH REDUCED GLYCOALKALOID AND METHOD FOR PRODUCING SAME

(71) Applicant: KANEKA CORPORATION, Osaka (JP)

(72) Inventors: Yozo NAGIRA, Hyogo (JP); Haruyasu HAMADA, Hyogo (JP); Akira ENDO, Hyogo (JP); Naoyuki UMEMOTO, Saitama (JP)

(21) Appl. No.: 18/568,982

(22) PCT Filed: Jun. 29, 2022
(86) PCT No.: PCT/JP2022/026124

§ 371 Date: Dec. 11, 2023

(30) Foreign Application Priority Data
Jul. 27, 2021 (JP) 2021-122711
Apr. 12, 2022 (JP) 2022-065686

(51) Int. Cl. A01H 6/82; C12N 9/16; C12N 9/78; C12N 15/82

(52) U.S. Cl. CPC A01H 6/827; C12N 9/16; C12N 9/78; C12N 15/8243

(57) ABSTRACT

A method for producing a genetically modified potato, which includes introducing deletion, insertion, or substitution into a CSLM gene of a potato. The genetically modified potato includes the CSLM gene into which deletion, insertion, or substitution is introduced.

### TECHNICAL FIELD

The present invention relates to a genetically modified potato with reduced glycoalkaloid, and a method for producing the same.

### BACKGROUND

Potatoes synthesize steroid glycoalkaloids (SGA) that are toxic substances, such as solanine and chaconine.

SGAs are accumulated in stem tubers and buds in stem tubers of potatoes. A low concentration of SGAs causes unpleasant tastes, such as bitterness. A high concentration of SGAs causes food poisoning. Therefore, it is important in breeding of potatoes to minimize an SGA content of potatoes.

Moreover, potatoes temporarily sustain growth or development during a few months of dormant period after harvesting. As budding starts after the dormancy, potatoes cannot be stored over a long period of time. Therefore, control of budding is an important task for the potato processing industry.

It has been demonstrated that knockdown of PGA1, PGA2, and 16DOX, which are genes associated with synthesis of solanine and chaconine, suppresses budding of potatoes (NPL 1 and NPL 2).

On the other hand, knockdown of SSR2 and PGA4, which are genes associated with synthesis of chaconine, cannot suppress budding of potatoes. The mechanism for controlling budding has not been understood.

Recently, it has been demonstrated that an SGA content is reduced by suppressing expression of the GAME15 gene using RNAi, but suppression of budding of potatoes has not been reported (PTL 1).

Genetically modified potatoes, where an SGA content is reduced, budding is delayed, and aging is inhibited by modification of endogenous genes of potatoes, and an efficient production method for such genetically modified potatoes have not been provided yet. Accordingly, there are growing demands for such genetically modified potatoes and an efficient production method thereof.

### CITATION LIST

### Patent Literature

PTL 1: US Patent Application Publication No. 2019/0059314

### Non-Patent Literature

NPL 1: Plant Physiology 2016 171:2458-2467
NPL 2: Plant Physiology 2017 175:120-133

### SUMMARY OF THE INVENTION

### Technical Problem

The present invention aims to solve the above-described various problems existing in the related art, and to achieve the following object. Specifically, an object of the present invention is to provide a genetically modified potato, where an SGA content is reduced, budding is delayed, and aging is inhibited by modification of endogenous genes of the potato, and an efficient method for producing such a genetically modified potato.

### Solution to Problem

The present inventors diligently conducted research to achieve the above-described object. As a result, the present inventors have found that a genetically modified potato, where an SGA content is reduced, budding is delayed, and aging is inhibited by modifying an endogenous gene of the potato, and an efficient method for producing such a genetically modified potato can be provided by applying a method for producing a genetically modified potato including introducing deletion, insertion, or substitution into a CSLM gene of the potato.

The present invention has been accomplished based on the above-described insight of the present inventors. Means for solving the above-described problems are as follows.

<1> A genetically modified potato, including:
a CSLM gene into which deletion, insertion, or substitution is introduced.

<2> A method for producing a genetically modified potato, the method including:
introducing deletion, insertion, or substitution into a CSLM gene of a potato.

<3> A genome editing method for a potato, the genome editing method including:
introducing deletion, insertion, or substitution into a CSLM gene of a potato using a genome editing agent.

<4> A composition for producing a genetically modified potato, the composition including:
a protein binding to a CSLM gene, a nucleic acid encoding the protein binding to the CSLM gene, a guide RNA targeting a CSLM gene, or a nucleic acid encoding the guide RNA targeting the CSLM gene; and
an enzyme of nucleic acid metabolism or a nucleic acid encoding the enzyme of nucleic acid metabolism.

<5> A method for determining a genetically modified potato, the method including:
determining whether a potato is a genetically modified potato or not based on the presence or absence of deletion, insertion, or substitution of a CSLM gene of the potato as an index.

### Advantageous Effects of Invention

According to the present invention, the above-described various problems existing in the related art can be solved, the above-described object can be achieved, and a genetically modified potato, where an SGA content is reduced, budding is delayed, and aging is inhibited by modification of endogenous genes of the potato, and an efficient method for producing such a genetically modified potato can be provided.

### BRIEF DESCRIPTION OF THE DRAWINGS

FIG. 1 is a photograph of stem tubers of a SSR2-silenced potato lineage depicting a state of budding after leaving the stem tubers in a dark place at 20° C.

FIG. 2 is a photograph of stem tubers of a PGA4-silenced potato lineage, depicting a state of budding after leaving the stem tubers in a dark place at 20° C.

FIG. 3 is a diagram illustrating a structure of a vector used for transformation.

FIG. 4 is a diagram depicting an alignment result (pSuehiro117 #343) of a base sequence near a target sequence of a CSLM gene for genome editing (the first line), and base sequences determined by cloning amplified fragments each including a genome-edited region from the genome-edited plant (the second line and below).

FIG. 5 is a diagram depicting an alignment result (pSuehiro117 #389) of a base sequence near a target sequence of a CSLM gene for genome editing (the first line), and base sequences determined by cloning amplified fragments each including a genome-edited region from the genome-edited plant (the second line and below).

FIG. 6 depicts the result of the quantitative analysis of the glycoalkaloid content of the genome-edited transformant.

FIG. 7 is a photograph of stem tubers of a CSLM genome-edited potato lineage, depicting a state of budding on the seventh week after leaving the stem tubers in a dark place at 20° C.

FIG. 8 is a graph depicting an average value of bud lengths and a total value of bud lengths in the seventh week after leaving the stem tubers of the CSLM genome-edited potato lineage in the dark place at 20° C.

FIG. 9 is a photograph of stem tubers of CSLM genome-edited potato linages, depicting a state of budding just after leaving stem tubers in a dark place at 20° C.

FIG. 10 is a photograph of stem tubers of CSLM genome-edited potato lineages, depicting a state of budding on the seventh week after leaving the stem tubers in a dark place at 20° C.

FIG. 11 is a photograph of stem tubers of CSLM genome-edited potato linages, depicting a state of budding on the ninth week after leaving the stem tubers in a dark place at 20° C.

FIG. 12 is a graph depicting an average value of bud lengths and a total value of bud lengths in the ninth week after leaving stem tubers of CSLM genome-edited potato lineages in a dark place at 20° C.

### DETAILED DESCRIPTION OF THE INVENTION

### (Genetically Modified Potato)

The genetically modified potato includes a cellulose synthase-like M gene (denoted as a CSLM gene hereinafter), in which deletion, insertion, or substitution is introduced. The synonym of the CSLM gene is the GAME15 gene.

The CSLM gene is not particularly limited, and may be appropriately selected according to the intended purpose. Examples of the CSLM gene include a base sequence encoding a protein having an amino acid sequence of SEQ ID NO: 1 or SEQ ID NO: 22, a base sequence encoding a homolog of a protein having an amino acid sequence of SEQ ID NO: 1 or SEQ ID NO: 22, and the like.

The homolog is not particularly limited, and may be appropriately selected according to the intended purpose. The homolog preferably has 80% or greater sequence identity, more preferably 85% or greater sequence identity, yet more preferably 90% or greater sequence identity, particularly preferably 95% or greater sequence identity, and the most preferably 99% or greater sequence identity, relative to the sequence of SEQ ID NO: 1 or SEQ ID NO: 22.

The base sequence encoding a protein having an amino acid sequence of SEQ ID NO: 1 or SEQ ID NO: 22 is not particularly limited, and may be appropriately selected according to the intended purpose. Examples of the base sequence encoding a protein having an amino acid sequence of SEQ ID NO: 1 or SEQ ID NO: 22 include a gene having a sequence of SEQ ID NO: 2 or SEQ ID NO: 23, a homolog of a gene having a sequence of SEQ ID NO: 2 or SEQ ID NO: 23, and the like.

The homolog is not particularly limited, and may be appropriately selected according to the intended purpose. The homolog preferably has 80% or greater sequence identity, more preferably 85% or greater sequence identity, yet more preferably 90% or greater sequence identity, particularly preferably 95% or greater sequence identity, and the most preferably 99% or greater sequence identity, relative to the sequence of SEQ ID NO: 2 or SEQ ID NO: 23.

The deletion, insertion, or substitution is not particularly limited, as long as the deletion, insertion, or substitution is artificially introduced. The deletion, insertion, or substitution may be appropriately selected according to the intended purpose. The deletion, insertion, or substitution may be any one of deletion, insertion, and substitution, or any combination between deletion, insertion, and substitution.

The deletion is not particularly limited, and may be appropriately selected according to the intended purpose. Examples of the deletion include deletion causing a frameshift mutation, deletion causing an in-frame mutation, and the like. Among the above-listed example, deletion causing a frameshift mutation is preferred.

The deletion causing the frameshift mutation can be achieved by introducing deletion of bases, where the number of the bases deleted is a number other than 3 or a multiple of 3.

The deletion causing the in-frame mutation can be achieved by introducing deletion of bases, where the number of the bases deleted is 3 or a multiple of 3.

In the deletion causing the in-frame mutation, the deleted site is preferably a site encoding an active site, an allosteric site, a protein-protein interaction site, etc., or a site adjacent to the above-mentioned site.

The insertion is not particularly limited, and may be appropriately selected according to the intended purpose. Examples of the insertion include insertion causing a frameshift mutation, insertion causing an in-frame mutation, and the like. Among the above-listed examples, insertion causing a frameshift mutation is preferred.

The insertion causing the frameshift mutation is achieved by introducing insertion of bases, where the number of the bases inserted is 3 or a multiple of 3.

The insertion causing the in-frame mutation is achieved by introducing insertion of bases, where the number of the bases inserted is 3 or a multiple of 3.

In the insertion causing in-frame mutation, the insertion site is preferably a site encoding an active site, an allosteric site, a protein-protein interaction site, etc., or adjacent to the above-mentioned site.

The substitution is not particularly limited, except that the substitution is nonsynonymous substitution. The substitution may be appropriately selected according to the intended purpose.

The substitution site is preferably a site encoding an active site, an allosteric site, a protein-protein interaction site, etc., or adjacent to the above-mentioned site.

The genetically modified potato preferably has the glycoalkaloid content that is reduced compared to a glycoalkaloid content of a genetically unmodified potato.

The reduction of the glycoalkaloid content is not particularly limited, and may be appropriately selected according to the intended purpose. The glycoalkaloid content of the genetically modified potato relative to a glycoalkaloid content of a genetically unmodified potato is preferably 50% or less, more preferably 30% or less, yet more preferably 20% or less, particularly preferably 10% or less, and the most preferably the detection limit or less.

The glycoalkaloid is not particularly limited, and may be appropriately selected according to the intended purpose. The glycoalkaloid is preferably solanine or chaconine.

Among the above-listed examples, the solanine content and chaconine content are preferably reduced compared to a solanine content and chaconine content of a genetically unmodified potato.

The reduction of the solanine content is not particularly limited, and may be appropriately selected according to the intended purpose. The solanine content of the genetically modified potato relative to a solanine content of a genetically unmodified potato is preferably 50% or less, more preferably 30% or less, yet more preferably 20% or less, particularly preferably 10% or less, and the most preferably the detection limit or less.

The reduction of the chaconine content is not particularly limited, and may be appropriately selected according to the intended purpose. The chaconine content of the genetically modified potato relative to a chaconine content of a genetically unmodified potato is preferably 50% or less, more preferably 30% or less, yet more preferably 20% or less, particularly preferably 10% or less, the most preferably the detection limit or less.

The glycoalkaloid content is measured in the following manner.

Leaves (about 100 mg) of a potato are frozen using liquid nitrogen, and the frozen leaves are crushed by a mixer mill (1/30 sec, 2 minutes). To the crushed leaves, 300 μL of methanol is added, followed by performing sonication for 10 minutes. Centrifugation (15,000 rpm, 10 minutes) is carried out, followed by collecting a supernatant. The above-described extraction process is repeated three times, followed by drying and solidifying the collected supernatant under reduced pressure. The obtained residues are again dissolved in 200 μL of methanol. To 20 μL of resulting solution, 180 μL methanol is added, followed by analyzing glycoalkaloid using LC-MS (product name: UPLC-ESI-MS ACQUITY, produced by Waters Corporation). As for a column, ACQUITY HSS T3 1.8 μm, a diameter of 2.1×100 mm (produced by Waters Corporation) is used. In the LC analysis, gradient elution is carried out with a mobile phase A: 0.1% formic acid in water and a mobile phase B: acetonitrile. The gradient elution is carried out while retaining from 90% A/10% B to 45% A/55% B for 0 to 30 minutes, from 45% A/55% B to 100% B for 30 minutes to 31 minutes, and 100% B for 31 minutes to 35 minutes. The quantification of glycoalkaloid is carried out in comparison with a standard.

A bud length of the genetically modified potato left in a dark place after the post-harvest dormant period is ended is preferably reduced compared to a bud length of a genetically unmodified potato.

The bud length is measured in the following manner.

The potato plant cultivated in a test tube is acclimated, and the potato plant is grown in culture soil (PRO-MIX BX, produced by Premier Tech) inside a closed greenhouse (23° C., 16 hours of lighting (photon flux density: 32 μE/m2s)/8 hours of no lighting) to harvest stem tubers. After harvesting, the stem tubers are stored for about 3 months at 4° C., followed by storing in a dark place at 20° C. for 7 weeks. The bud length of each of buds of 5 mm or longer is measured to determine an average value of the bud lengths of buds of each of the stem tubers or a total value of the bud lengths of buds of each of the stem tubers.

The measurement of the bud length is performed on at least three stem tubers, and the arithmetic mean of the measured values is determined.

The reduction of the bud length (the average value of the bud lengths of the buds of each of the stem tubers) is not particularly limited, and may be appropriately selected according to the intended purpose. The budding length of the genetically modified potato relative to a budding length of a genetically unmodified potato is preferably 80% or less, more preferably 70% or less, yet more preferably 50% or less, particularly preferably 30% or less, and the most preferably 20% or less.

The reduction of the bud length (the total value of the bud lengths of the buds of each of the stem tubers) is not particularly limited, and may be appropriately selected according to the intended purpose. The budding length of the genetically modified potato relative to a budding length of a genetically unmodified potato is preferably 80% or less, more preferably 70% or less, yet more preferably 50% or less, particularly preferably 30% or less, and the most preferably 20% or less.

Regarding the reduction of the bud length, either the average value of the bud lengths of the buds of each of the stem tubers or the total value of the bud lengths of the buds of each of the stem tubers is preferably reduced, and more preferably, both the average value of the bud lengths of the buds of each of the stem tubers and the total value of the bud lengths of the buds of each of the stem tubers are reduced.

### (Method for Producing Genetically Modified Potato)

The method for producing a genetically modified potato includes introducing deletion, insertion, or substitution into a CSLM gene of a potato, and may further include other steps.

The introducing deletion, insertion, or substitution into a CSLM gene of a potato is not particularly limited, and may be appropriately selected according to the intended purpose. Examples of the step include a method using genome editing, and the like.

The method using genome editing is not particularly limited, and may be appropriately selected according to the intended purpose. Examples of the method include a method where deletion, insertion, or substitution is introduced into a CSLM gene of a potato using a genome editing agent, and the like.

The method of introducing is not particularly limited, and may be appropriately selected according to the intended purpose. Examples of the method include: a method where a genome editing agent is introduced into Agrobacterium tumefaciens, and a potato is infected with the Agrobacterium tumefaciens; a method where particles each coated with a genome editing agent are fired into a potato; and the like.

The genome editing agent is not particularly limited, and may be appropriately selected according to the intended purpose. Examples of the genome editing agent include a combination of a protein binding to a CSLM gene, a nucleic acid encoding the protein binding to the CSLM gene, a guide RNA targeting a CSLM gene, or a nucleic acid encoding the guide RNA targeting the CSLM gene with an enzyme of nucleic acid metabolism or a nucleic acid encoding the enzyme of nucleic acid metabolism, and the like. The genome editing agent may include other nucleic acids, or other proteins.

The genome editing agent may be a gene editing vector.

The gene editing vector is not particularly limited, and may be appropriately selected according to the intended purpose. Examples of the gene editing vector include a vector including a binary vector as a base, and the like.

A sequence included in the gene editing vector is not particularly limited, and may be appropriately selected according to the intended purpose. Examples of the sequence include a combination of a sequence encoding a protein binding to a CSLM gene or a nucleic acid encoding a guide RNA targeting a CSLM gene with a nucleic acid encoding an enzyme of nucleic acid metabolism, and the like. The sequence may further include other sequences.

A partial sequence of a CSLM gene identified by the protein binding to the CSLM gene is not particularly limited, and may be appropriately selected according to the intended purpose. Examples of the partial sequence include: TGCAAAGATTCCGATCTaccaccaattgacgtAATGGTATTCACTGCCA (SEQ ID NO: 3); TGCAAAGATTCCGATCT (SEQ ID NO: 4) and AATGGTATTCACTGCCA (SEQ ID NO: 5) that are uppercase regions of the sequence TGCAAAGATTCCGATCTaccaccaattgacgtAATGGTATTCACTGCCA (SEQ ID NO: 3); and the like.

The lower limit of the length of the guide RNA is not particularly limited, and may be appropriately selected according to the intended purpose. The length of the guide RNA is preferably 15 nucleotides or longer, more preferably 16 nucleotides or longer, yet more preferably 17 nucleotides or longer, particularly preferably 18 nucleotides or longer, yet particularly preferably 19 nucleotides or longer, and the most preferably 20 nucleotides or longer.

The upper limit of the length of the guide RNA is not particularly limited, and may be appropriately selected according to the intended purpose. The length of the guide RNA is preferably 30 nucleotides or shorter, more preferably 25 nucleotides or shorter, more preferably 22 nucleotides or shorter, and particularly preferably 20 nucleotides or shorter.

The guide RNA may include a guide sequence fused to a tracer sequence.

The CSLM gene may include a mutation between cleavage sites, where the mutation may be caused by introducing insertion of or substitution with a foreign DNA by recombination.

The enzyme of nucleic acid metabolism is not particularly limited, and may be appropriately selected according to the intended purpose. Examples of the enzyme of nucleic acid metabolism include nuclease, deaminase, and the like. The enzyme of nucleic acid metabolism may include one or more nuclear localization signals (NLS).

The nuclease is not particularly limited, and may be appropriately selected according to the intended purpose. Examples of the nuclease include CAS nucleases of CRISPR-CAS system, zinc finger nucleases, proteins expressing zinc finger nucleases, TAL effector nucleases (TALEN), TARGET AID, meganucleases, and the like. Moreover, the nuclease may be a nuclease derived from a different biological species. For example, genes of animals, plants, microorganisms, viruses, or the like, or artificially synthesized genes may be used.

The CAS nuclease is not particularly limited, and may be appropriately selected according to the intended purpose. Examples of the CAS nuclease include Type I CRISPR enzymes, Type II CRISPR enzymes, Type III CRISPR enzymes, and the like. Among the above-listed examples, Cas9 that is a Type II CRISPR enzyme is preferred.

The codon optimization of the Cas nuclease may be performed for expression in eukaryotic cells. The Cas nuclease may cause local cleavage of one or two strands of a target sequence.

The Cas9 is not particularly limited, and may be appropriately selected according to the intended purpose. Examples of Cas9 include Cas9 of Streptococcus pneumoniae, Cas9 of Streptococcus pyogenes, Cas9 of Streptococcus thermophilus, Cas9 of Staphylococcus aureus, and the like. Among the above-listed examples, Cas9 of Streptococcus pyogenes is preferred. Moreover, the Cas9 may be a Cas9 mutant derived from any of the foregoing organisms, or a Cas9-D10A mutant, which is known to function as nickase (a DNA cleaving enzyme that introduces a nick only in one of DNA strands), or a Cas9 homolog or ortholog.

A nuclease domain in the TALEN is not particularly limited, and may be appropriately selected according to the intended purpose. Examples of the nuclease domain include nuclease domains derived from Type II restriction endonucleases, such as HhaI, HindIII, Nod, BbvCI, EcoRI, BglI, AlwI, and FokI, and the like.

Among the above-listed examples, FokI is preferred.

The TAL effector DNA-binding domain in the TALEN is not particularly limited, and may be appropriately selected according to the intended purpose. Examples of the TAL effector DNA-binding domain include TAL effector DNA-binding domains derived from Xanthomonas plant pathogen.

The deaminase is not particularly limited, except that the deaminase exhibits a deaminase activity. The deaminase may be appropriately selected according to the intended purpose. The deaminase can be used by adding the deaminase to a CRISPR-CAS system from which a nuclease activity is removed.

The above-mentioned other sequences are not particularly limited, and may be appropriately selected according to the intended purpose. Examples of the above-described other sequences include promoters, enhancers, insulators, introns, terminators, poly(A) addition signals, selectable marker genes, and the like.

The promoter may not be a promoter derived from a plant, as long as the promoter functions on a potato to structurally induce expression, or the promoter is a DNA capable of inducing expression in a specific tissue of a potato or at a specific growth stage. Specific examples of the promoter include cauliflower mosaic virus (CaMV) 35S promoters, E12-35S omega promoters, nopaline synthase gene promoters (Pnos), maize ubiquitin promoters, rice actin promoters, tobacco PR protein promoters, ADH promoters, RuBisco promoters, and the like.

The translation efficiency can be enhanced by using a sequence for enhancing a translation activity, such as an omega sequence of a tobacco mosaic virus. Moreover, proteins may be translated from a plurality of coding regions by inserting an internal ribosomal entry site (IRES) serving as a translation initiation region at the 3′-downstream side of the promoter and the 5′-upstream side of the start codon.

The terminator is not limited, as long as the terminator can terminate the transcription of the gene transcribed by the promoter and has a sequence including a poly(A) addition signal. Examples of the terminator include terminators of nopaline synthase (NOS) genes, terminators of octopine synthase (OCS) genes, CaMV 35S terminators, and the like.

Examples of the selectable marker gene include herbicide resistant genes (e.g., the third introns of phytoene desaturase genes (AT4g14210), bialophos resistant genes, glyphosate resistant genes (EPSPS), sulfonylurea-based herbicide resistant genes (ALS), etc.), drug resistant genes (e.g., tetracycline resistant genes, amipicillin resistant genes, kanamycin resistant genes, hygromycin resistant genes, spectinomycin resistant genes, chloramphenicol resistant genes, neomycin resistant genes, etc.), fluorescent or luminescent reporter genes (e.g., luciferase, β-galactosidase, β-glucuronidase (GUS), green fluorescent protein (GFP), etc.), and enzyme genes, such as neomycin phosphotransferase II (NPT II) and dihydrofolate reductase, and the like.

A method of introducing the genome editing vector into Agrobacterium tumefaciens is not particularly limited, and may be appropriately selected according to the intended purpose. Examples of the method include a freeze-thawing method, an electroporation method, and the like.

A method of infecting the potato with the Agrobacterium tumefaciens is not particularly limited, and may be appropriately selected according to the intended purpose. Examples of the method include a method where a stem or microtuber of a potato is infected with the Agrobacterium tumefaciens, and the like.

A method of firing the particles each coated with the genome editing agent into the potato is not particularly limited, and may be appropriately selected according to the intended purpose. Examples of the method include a method where particles each coated with the genome editing agent are fired into a plumule or axillary bud of a potato, and the like.

The particles are not particularly limited, and may be appropriately selected according to the intended purpose. Considering enhancement of penetration power into cells at the time of firing, the particles are preferably particles that have high specific gravity, are chemically inert, and are unlikely to harm organisms. Examples of the particles include metal particles, ceramic particles, glass particles, and the like.

The metal particles are not particularly limited, and may be appropriately selected according to the intended purpose. Examples of the metal particles include free metal particles, alloy particles, and the like.

The free metal particles are preferably gold particles, tungsten particles, and the like.

The lower limit of the mean particle diameter of the particles is not particularly limited, and may be appropriately selected according to the intended purpose. The lower limit of the mean particle diameter of the particles is preferably 0.3 μm or greater, more preferably 0.4 μm or greater, yet more preferably 0.5 μm or greater, and particularly preferably 0.6 μm or greater.

The upper limit of the mean particle diameter of the particles is not particularly limited, and may be appropriately selected according to the intended purpose. The upper limit of the mean particle diameter of the particles is preferably 1.5 μm or smaller, more preferably 1.4 μm or smaller, yet more preferably 1.3 μm or smaller, particularly preferably 1.2 μm or smaller, particularly preferably 1.1 μm, and the most preferably 1.0 μm or smaller.

The mean particle diameter is not particularly limited, and may be appropriately selected according to the intended purpose. Examples of the mean particle diameter include a number average particle diameter, and the like.

Shapes of the particles are not particularly limited, and may be appropriately selected according to the intended purpose. Examples of the shapes include spheres, cubes, rods, plates, and the like. Among the above-listed examples, spheres are preferred.

A method of coating is not particularly limited, and may be appropriately selected according to the intended purpose. Examples of the method include a method where the particles are washed and sterilized, the resulting particles, a nucleic acid (e.g., a recombinant vector, a linear DNA, RNA, etc.) and/or a protein, CaCl₂, spermidine, and the like are added together while stirring the mixture with a vortex mixer or the like so that the particles are each coated (covered) with the nucleic acid and/or protein, and the resulting coated particles are washed with ethanol or phosphate-buffered saline (PBS, etc.), and the like.

The particles may be applied onto a microcarrier film as homogeneously as possible using a micropipette or the like, followed by drying in a sterile environment, such as a clean bench. In the case of particles each coated with a protein, a hydrophilic microcarrier film is preferably used.

The hydrophilic microcarrier film may be prepared by bonding an hydrophilic film to a microcarrier film, or applying hydrophilic coating to a microcarrier film.

Examples of the method of imparting hydrophilicity to a film include a method where a surfactant, a photocatalyst, or a hydrophilic polymer is used, and the like.

The hydrophilic polymer is not particularly limited, and may be appropriately selected according to the intended purpose. Examples of the hydrophilic polymer include polymers of hydrophilic monomers (e.g., polyethylene glycol, hydroxyethyl methacrylate, hydroxypropyl methacrylate, dihydroxyethyl methacrylate, diethylene glycol methacrylate, triethylene glycol methacrylate, polyethylene glycol methacrylate, vinyl pyrrolidone, acrylic acid, acrylamide, dimethyl acrylamide, glucoxyoxyethyl methacrylate, 3-sulfopropylmethacryloxyethyldimethyl ammonium betaine, 2-methacryloyloxyethyl phosphorylcoline, 1-carboxydimethylmethacryloyloxyethylmethane ammonium, etc.), and the like.

The coverage rate of the particles is not particularly limited, and may be appropriately selected according to the intended purpose. The entire surface of each of the particles may be coated or part of the surface of each of the particles may be coated.

The means for firing the particles is not particularly limited, except that particles can be fired into plant cells. Examples of the means for firing include a particle gun (gene gun) according to a particle gun method, and the like. In view of transfer efficiency, a method where particles are introduced into a plumule or axillary bud according to the particle gun method is preferred.

A method of introducing the particles into a plumule or axillary bud according to the particle gun method is not particularly limited, and may be appropriately selected according to the intended purpose. Examples of the method include a method where a microcarrier film to which the particles are coated, and a plate on which a shoot apical meristem of a plumule or axillary bud of a target is placed are set in a particle gun device, and high-pressure gas is ejected from a gas acceleration tube towards the microcarrier film, and the like. The microcarrier film is stopped by a stopping plate, but the particles coated on the microcarrier film pass through the stopping plate to penetrate into the target set below the stopping plate to thereby transfer the target gene.

The high-pressure gas is not particularly limited, and may be appropriately selected according to the intended purpose. Examples of the high-pressure gas include helium and the like.

The particle gun device is not particularly limited, and may be appropriately selected according to the intended purpose. Examples of the particle gun device include Biolistic (registered trademark) PDS-1000/He Particle Delivery System (BIO-RAD), and the like.

The upper limit of the distance between the stopping plate and the shoot apical meristem serving as a target is not particularly limited, and may be appropriately selected according to the intended purpose. The upper limit of the distance is preferably 9 cm or less, more preferably 8 cm or less, yet more preferably 7 cm or less, and particularly preferably 6 cm or less.

The lower limit of the distance between the stopping plate and the shoot apical meristem that is a target is not particularly limited, and may be appropriately selected according to the intended purpose. The lower limit of the distance is preferably 2 cm or greater, more preferably 3 cm or greater, and yet more preferably 4 cm or greater.

The gas pressure of the particle gun device is not particularly limited, and may be appropriately selected according to the intended purpose. The gas pressure is preferably from 1,100 psi to 1,600 psi, more preferably from 1,200 psi to 1,500 psi.

The lower limit of the number of times the particles are fired into the shoot apical meristem is not particularly limited, and may be appropriately selected according to the intended purpose. The lower limit is preferably twice or more, more preferably three times or more, and yet more preferably four times or more.

The upper limit of the number of times the particles are fired into the shoot apical meristem is not particularly limited, and may be appropriately selected according to the intended purpose. The upper limit is preferably 20 times or less, more preferably 15 times or less, and yet more preferably 10 times or less.

Examples of the above-mentioned other steps include selecting a potato, in which deletion, insertion, or substitution is introduced into a CSLM gene of the potato, and the like.

The selecting is not particularly limited, and may be appropriately selected according to the intended purpose. Examples of the selecting include a method where selection is carried out using a drug resistant gene.

### (Genome Editing Method for Potato)

The genome editing method for a potato includes introducing deletion, insertion, or substitution into a CSLM gene of a potato using a genome editing agent. The genome editing method for the potato may further include other steps.

The introducing deletion, insertion, or substitution into a CSLM gene of a potato using a genome editing agent is as described in the above-described method of introducing deletion, insertion, or substitution into a CSLM gene of a potato using the genome editing agent.

### (Composition for Producing Genetically Modified Potato)

The composition for producing a genetically modified potato includes: a protein binding to a CSLM gene, a nucleic acid encoding the protein binding to the CSLM gene, a guide RNA targeting a CSLM gene, or a nucleic acid encoding the guide PNA targeting the CSLM gene; and an enzyme of nucleic acid metabolism or a nucleic acid encoding the enzyme of nucleic acid metabolism. The composition may further include other components.

The protein binding to the CSLM gene, the nucleic acid encoding the protein binding to the CSLM gene, the guide RNA targeting the CSLM gene, or the nucleic acid encoding the guide RNA targeting the CSLM gene, and the enzyme of nucleic acid metabolism or the nucleic acid encoding the enzyme of nucleic acid metabolism are as described above.

### (Method for Determining Genetically Modified Potato)

The method for determining a genetically modified potato includes determining whether a potato is a genetically modified potato or not based on the presence or absence of deletion, insertion, or substitution of a CSLM gene of the potato as an index. The method may further include other steps.

The determining whether a potato is a genetically modified potato or not based on the presence or absence of deletion, insertion, or substitution of a CSLM gene of the potato as an index is not particularly limited, and may be appropriately selected according to the intended purpose. Examples of the step include a method where a sequence of a CSLM gene of a potato is analyzed to compare with a DNA sequence of SEQ ID NO: 2 or SEQ

ID NO: 23. According to the method for determining a genetically modified potato, whether or not a potato in question is a genetically modified potato, where an SGA content is reduced, budding is delayed, and aging is inhibited by modification of an endogenous gene of the potato, can be efficiently determined.

### EXAMPLES

Examples of the present invention will be described hereinafter, but the present invention is not limited to these Examples in any way.

### Comparative Example 1: Production of Stem Tubers from SSR2-Silenced Potato Lineage and Examination of Budding

A partial segment (269 bp) from a cDNA sequence (SEQ ID NO: 6) of SSR2 was amplified by PCR using the following primers,

Primer 1 (SEQ ID NO: 7):
GAGCTCTAGACCCTAGGAGGAAGATCCAG;
and
Primer 2 (SEQ ID NO: 8):
GGATCCATATGCGTTTCTCATTCCAACAACA.

The two fragments (the sense strand of the target sequence and the antisense strand of the target sequence) and the third intron sequence (a loop sequence) of Arabidopsis thaliana At4g14210 gene were inserted between the CaMV35S promoter and the CaMV35S terminator in the T-DNA region to thereby construct an RNAi binary vector pKT251 for SSR2 gene silencing. Using the Agrobacterium tumefaciens strain to which the above-described plasmid was transferred (GV3101mp90 (strain with pMP90RK disclosed in Mol Gen Genet (1986) 204:383-396) with pKT251), transformation of Sassy (S. tuberosum cv. Sassy) was carried out according to the method disclosed in the literature, Monnma, T. (1990). Recent study for genetic engineering of soybean glycinin gene. Plant Tissue Cult. Lett. 7:57-63.

The cultured seedling having the genome to which a kanamycin resistant gene was inserted was selected by a genomic analysis with PCR using the following primers, Primer 3 (SEQ ID NO: 9): TAAAGCACGAGGAAGCGGT; and Primer 4 (SEQ ID NO: 10): GCACAACAGACAATCGGCT. Moreover, RT-PCR analysis was performed using the following primers, Primer 5 (SEQ ID NO: 11): CTCTGCTCAAAGCCACACAA; and Primer 6 (SEQ ID NO: 12): TCAATTCGCAGGTTCATCAG, to acquire SSR2-RNAi potato lineages (#2, #10, #11, and #12), in which expression of the SSR2 gene was extremely low or the SSR2 gene was not expressed.

The potato plant of each of the SSR2-RNAi potato lineages (#2, #10, #11, #12, and non-transformant (NT) of Sassy), which was cultivated in a test tube, was acclimated, and the potato plant was grown in culture soil (PRO-MIX BX, produced by Premier Tech) inside a closed greenhouse (23° C., 16 hours of lighting (photon flux density: 32 μE/m2s)/8 hours of no lighting) to harvest stem tubers. After harvesting, the stem tubers were stored for about 3 months at 4° C. After ending the dormant period, budding was observed in a dark place at 20° C. FIG. 1 is a photograph depicting the state of budding on the 13th week from the start of the test in the dark place at 20° C.

### Comparative Example 2: Production of Stem Tubers from PGA4-Silenced Potato Lineage and Examination of Budding

A partial segment (394 bp) from a cDNA sequence (SEQ ID NO: 13) of PGA4 was amplified by PCR using the following primers,

Primer 7 (SEQ ID NO: 14):
GAGCTCTAGATATTTGATTTGCCACCTCCAT;
and
Primer 8 (SEQ ID NO: 15):
GGATCCATATGCTTACAAGCACAGCACCAA.

Using the fragments, an RNAi binary vector pKT250 for PGA4 gene silencing was constructed in the same manner as in Comparative Example 1. Using the Agrobacterium tumefaciens strain to which the above-described plasmid was transferred (GV3101mp90 (strain with pMP90RK disclosed in Mol Gen Genet (1986) 204:383-396) with pKT250), transformation of Sassy (S. tuberosum cv. Sassy) was carried out according to the method disclosed in NPL 1 “Gene-Silenced Potatoes.”

The cultured seedling having the genome to which a kanamycin resistant gene was inserted was selected by a genomic analysis with PCR using the following primers, Primer 3 (SEQ ID NO: 9) and Primer 4 (SEQ ID NO: 10). Moreover, RT-PCR analysis was performed using the following primers, Primer 9 (SEQ ID NO: 16): TGGGGTGTTGGTACATATTTTG and Primer 10 (SEQ ID NO: 17): TTCCTCTTTGGCTTTCTCCA, to acquire PGA4-RNAi potato lineages (#9, #11, #16, and #22), in which expression of the PGA4 gene was extremely low or the PGA4 gene was not expressed.

The potato plant of each of the PGA4-RNAi potato lineages (#9, #11, #16, #22, and non-transformant (NT) of Sassy), which was cultivated in a test tube, was acclimated, and the potato plant was grown in culture soil (PRO-MIX BX, produced by Premier Tech) inside a closed greenhouse (23° C., 16 hours of lighting (photon flux density: 32 μE/m2s)/8 hours of no lighting) to harvest stem tubers.

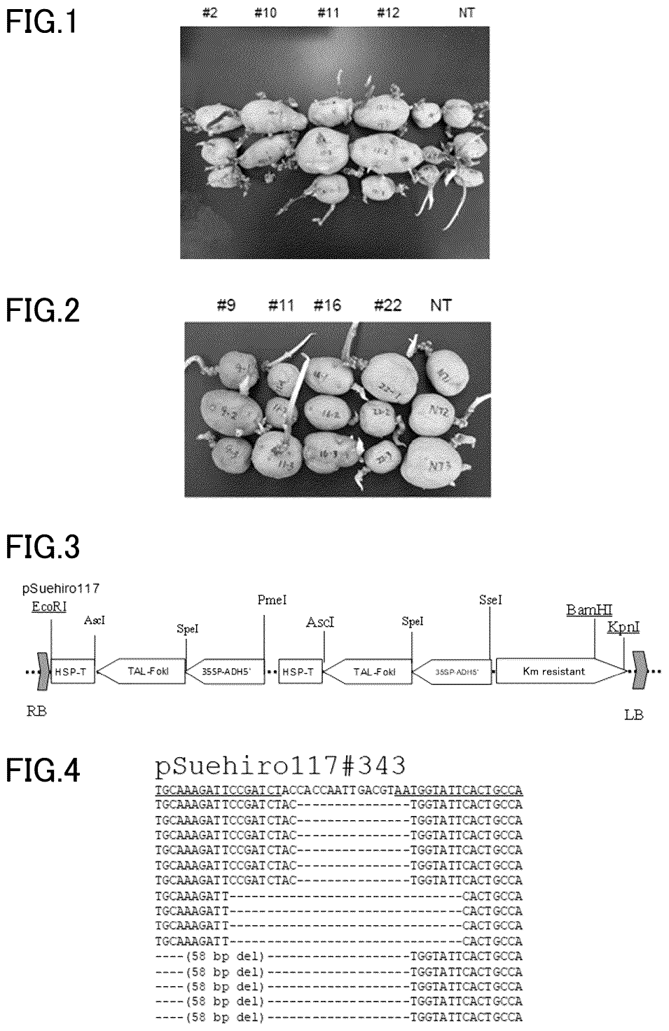

After harvesting, the stem tubers were stored for about 3 months at 4° C. After ending the dormant period, budding was observed in a dark place at 20° C. FIG. 2 is a photograph depicting the state of budding on the 14^{th} week from the start of the test in the dark place at 20° C.

### Example 1: Acquisition of Potato CSLM Gene

It has been known that there is a cellulose synthase-like M gene (CSLM gene) Sotub07g016530.1.1, which is co-expressed with a glycoalkaloid biosynthesis gene next to chromosome 12 of the glycoalkaloid biosynthesis gene, in the genome database (Spud DB: http://solanaceae.plantbiology.msu.edu/index.shtml) of the potato (S. tuberosum) (Itkin et al., Science 2013 341:175-9).

Note that, currently, “Soltu.DM.07G014150.1” (SEQ ID NO: 23) is registered instead of the above-mentioned “Sotub07g016530.1.1” (SEQ ID NO: 2) in the genome database (Spud DB: http://solanaceae.plantbiology.msu.edu/index.shtml) of the potato (S. tuberosum). Compared to the “Sotub07g016530.1.1,” “Soltu.DM.07G014150.1” has the start codon whose location is 12 bases upstream, and the location of the stop codon of the “Soltu.DM.07G014150.1” was changed to the downstream direction. The sequence of “Soltu.DM.07G014150.1” corresponding to “Sotub07g016530.1.1” has 99% sequence identity of “Sotub07g016530.1.1” so that the both sequences can be handled as the same sequence.

As an expression analysis of the CSLM gene, a gene expression level (fpkm: fragments per kilobase of exon per million reads mapped, which is one of general indexes for expression levels) of SRX3127474:Solanum tuberosum, Atlantic, leaf, RNA-Seq, which was the NGS data registered on the database of NCBI was analyzed using Trinity (https://github.com/trinityrnaseq/trinityrnaseq/wiki), Bowtie (http://bowtie-bio.sourceforge.net/index.shtml), DEGseq (Original site), and eXpress (Original site). As a result, the fpkm value was calculated as 236.88, which suggested that the CSLM gene was associated with glycoalkaloid biosynthesis.

### Example 2: Acquisition of Genome Sequence of Potato CSLM Gene

In order to determine the genome sequence of the CSLM gene of “Sassy” that was the variety of the potato provided to the test, PCR (30 cycles, using PrimeSTAR (registered trademark) produced by TAKARA BIO INC.) of the genome DNA of “Sassy” (produced by Germicopa) was carried out based on the sequence of the Sotub07g016530.1.1, using the synthesized primers GTTTAAATTCGAAAACATTCTGAGT:U1153 (SEQ ID NO: 18) and ATGACAAAGGACATCCTCCA:U1154 (SEQ ID NO: 19) at the annealing temperature of 55° C.

The obtained PCR amplification products were cloned into a pENTR/D-TOPO (registered trademark) vector (produced by Thermo Fisher Scientific Inc.) to acquire the gene segments, and absence of polymorphisms of

(SEQ ID NO: 3)
TGCAAAGATTCCGATCTaccaccaattgacgtAATGGTATTCACTGCCA

of the first exon was confirmed.

### Example 3: Production of Genome Editing Vector for Potato CSLM Gene

Based on the genome DNA sequence determined in Example 2, a platinum TALEN vector pSuehiro117 identifying the uppercase regions of the sequence of

(SEQ ID NO: 3)
TGCAAAGATTCCGATCTaccaccaattgacgtAATGGTATTCACTGCCA

was prepared according to the method proposed by Yasumoto et al. (Plant Biotechnol 2019 36:167-173) (FIG. 3, vector pSuehiro117).

FIG. 3 illustrates a right border (RB) and left border (LB) of the gene segment of T-DNA for gene transfer, and the internal structure inside the borders. Each of the boxes in FIG. 3 represents an exon.

### Example 4: Production of Transformant of Potato CSLM Gene

The vector produced in Example 3 was introduced into the Agrobacterium tumefaciens GV3101mp90 strain (strain with pMP90RK disclosed in Mol Gen Genet (1986) 204:383-396) by a freeze-thawing method. The Agrobacterium tumefaciens GV3110mp90 strain including the vector was subjected to shake culture in a kanamycin (50 ppm)-containing YEB liquid culture medium [5 g/L of beef extract, 1 g/L of yeast extract, 5 g/L of peptone, 5 g/L of sucrose, 2 mM of magnesium sulfate (pH 7.2)] for 12 hours at 28° C. Centrifugation of the liquid culture medium (1.5 mL) was performed for 3 minutes at 10,000 rpm to harvest, followed by re-suspending in 1.5 mL of a 3% sucrose-containing MS culture medium (Physiol. Plant. 1962 15:473-497), to thereby prepare an inoculum.

The stem of the variety of potato “Sassy” cultivated in a test tube was cut into a piece in the length of 3 mm to 5 mm, which did not include nodes, and was provided as a material for infecting with Agrobacterium tumefaciens. The prepared material was immersed in the above-described inoculum of Agrobacterium tumefaciens, followed by placing the material on sterilized filter paper to remove excess Agrobacterium tumefaciens. The material was then placed on an MS culture medium (including 2 ppm of Zeatin, 0.05 ppm of IAA, 100 μM of acetosyringone, and 0.8% of agar) in a petri dish, followed by cultivating for 3 days at 25° C. under 16 hours of lighting (photon flux density: 32 μE/m2s)/8 hours of no lighting.

Subsequently, the cultivated material was further cultivated for one week on a culture medium including 250 ppm of carbenicillin instead of acetosyringone. Thereafter, the cultivated material was further placed on a culture medium including 50 ppm of kanamycin, followed by subculturing every two weeks. During this period, adventitious buds were formed to grow shoots.

The extended buds were placed on an MS culture medium that included 250 ppm of carbenicillin and 100 ppm of kanamycin but not a plant growth regulator.

Among the grown kanamycin resistant plants, the plant including the kanamycin resistant gene were detected by performing PCR (conditions: 95° C. for 5 minutes, (95° C. for 30 seconds, 55′C for 30 seconds, and 72′C for 1 minute) 30 times, and 72° C. for 10 minutes) to confirm that the redifferentiated plant was a transformed plant.

As primers specifically amplifying the sequence of the kanamycin resistant gene, NP2: TAAAGCACGAGGAAGCGGT (SEQ ID NO: 20) and NP3: GCACAACAGACAATCGGCT (SEQ ID NO: 21) were used.

As described above, the transformed plant of each of the pSuehiro117 #343 lineage and the pSuehiro117 #389 lineage of the potato, into which pSuehiro117 was transferred, was acquired.

### Example 5: Examination of Potato Lineage in which Genome of Potato CSLM Gene was Edited

A heteroduplex mobility assay (HMA: Heteroduplex Mobility Assay) was used to evaluate whether or not the genome was site-specifically edited in the obtained lineages.

PCR (35 cycles, using TakaraTaq produced by TAKARA BIO INC.) was performed using the above-mentioned primers (SEQ ID NOs: 18 and 19), between which a target sequence of the CSLM gene was located, at an annealing temperature of 55° C., and analysis was performed by a microchip electrophoresis system “MultiNA” (produced by Shimadzu Corporation).

In the heteroduplex mobility assay (HMA), the results demonstrating a plurality of bands in comparison with the control confirmed that pSuehiro117 #343 and pSuehiro117 #389 were the genome-edited plants.

In order to confirm the deletion of the base sequence achieved by the genome editing, the DNA segments, which were amplified by the gRNA, of the genome of the leaves of the transformant of each of pSuehiro117 #343 and pSuehiro117 #389 were cloned into TOPO (registered trademark) TA Cloning (registered trademark) Kit for Sequencing (produced by Thermo Fisher Scientific Inc.) to acquire gene fragments.

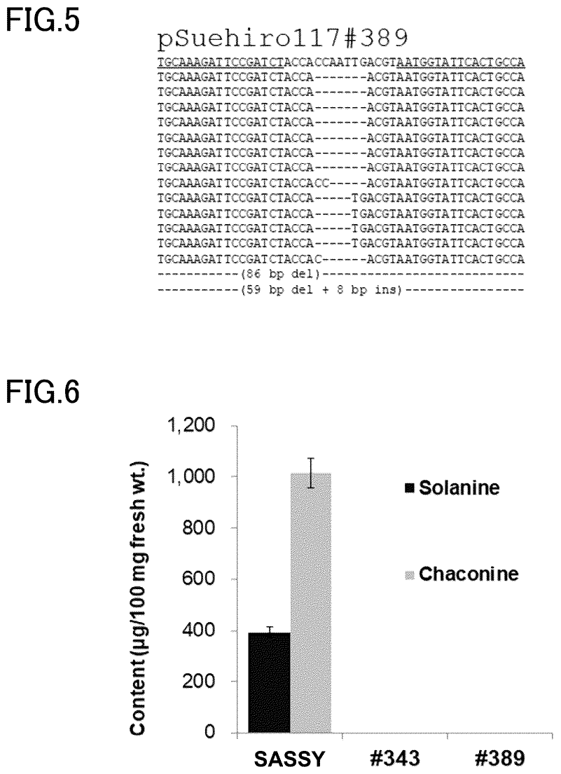

About 16 base sequences were determined, and it was confirmed that the genome editing occurred (FIG. 4: pSuehiro117 #343, FIG. 5: pSuehiro117 #389).

In FIGS. 4 and 5, “-” denotes deletion.

It was confirmed from the results above that both lineages were lineages that did not have an unmodified CSLM gene.

### Example 6: Determination of Glycoalkaloid Content of CSLM Genome-Edited Potato Lineage

Leaves (about 100 mg) of the obtained plant were frozen using liquid nitrogen, and the frozen leaves were crushed by a mixer mill (1/30 sec, 2 minutes).

To the crushed leaves, 300 μL of methanol was added, followed by performing sonication for 10 minutes. Centrifugation (15,000 rpm, 10 minutes) was carried out, followed by collecting a supernatant. The above-described extraction process was repeated three times, followed by drying and solidifying the collected supernatant under reduced pressure. The obtained residues were again dissolved in 200 μL of methanol.

To 20 μL of resulting solution, 180 μL methanol was added, followed by analyzing glycoalkaloid using LC-MS (product name: UPLC-ESI-MS ACQUITY, produced by Waters Corporation). As for a column, ACQUITY HSS T3 1.8 μm, a diameter of 2.1×100 mm (produced by Waters Corporation) was used.

In the LC analysis, gradient elution was carried out with a mobile phase A: 0.1% formic acid in water and a mobile phase B: acetonitrile. The gradient elution was carried out while retaining from 90% A/10% B to 45% A/55% B for 0 to 30 minutes, from 45% A/55% B to 100% B for 30 minutes to 31 minutes, and 100% B for 31 minutes to 35 minutes.

The quantification of solanine and chaconine was carried out in comparison with standards. As a result, generation of glycoalkaloid was not confirmed in the transformants of pSuehiro117 #343 and pSuehiro117 #389 as depicted in FIG. 6.

### Example 7: Production of Stem Tubers from Potato CSLM Genome-Edited Potato Lineage and Examination of Budding

The potato plant (each of non-transformant (NT) of Sassy, transformant of pSuehiro117 #343, and transformant of pSuehiro117 #389) cultivated in a test tube was acclimated, and the potato plant was grown in culture soil (PRO-MIX BX, produced by Premier Tech) inside a closed greenhouse (23° C., 16 hours of lighting (photon flux density: 32 μE/m2s)/8 hours of no lighting) to harvest stem tubers.

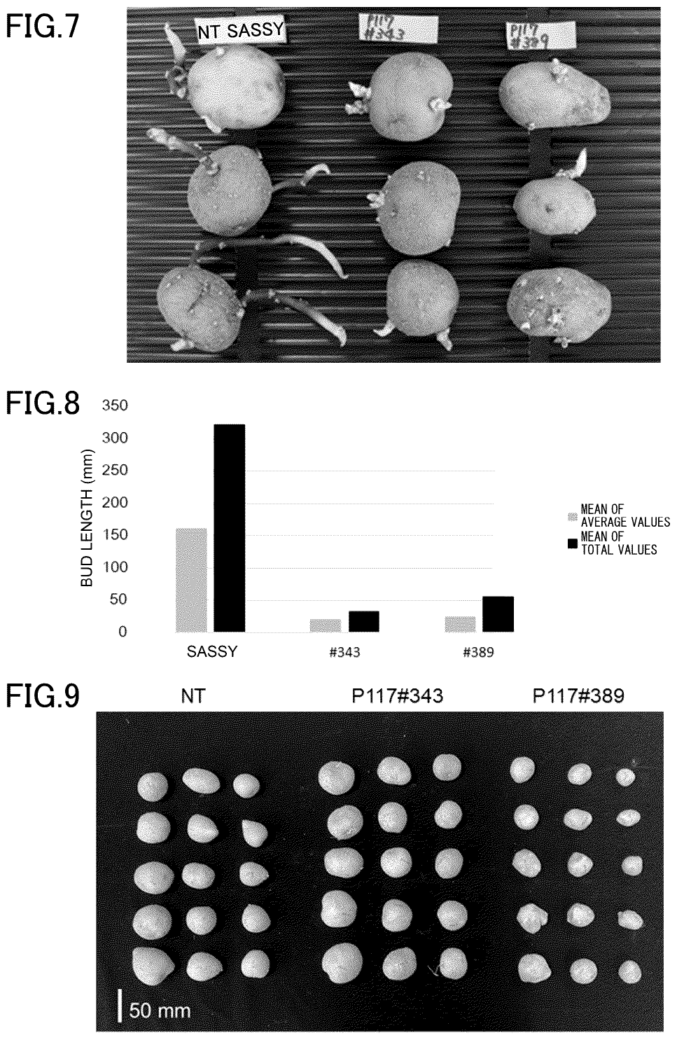

After harvesting, the stem tubers were stored for about 3 months at 4° C. After ending the dormant period, budding in three stem tubers from each group was observed in a dark place at 20° C. FIG. 7 is a photograph depicting the state of budding on the seventh week from the start of the test in the dark place at 20° C. (left: non-transformant (NT) of Sassy, center: transformant of pSuehiro117 #343, right: transformant of pSuehiro117 #389).

The bud lengths were measured. The measured values, the average value of the bud lengths, and the total value of the bud lengths are presented in Table 1 and FIG. 8. The softness (aging) of the stem tubers was observed. The results are presented in Table 1.

TABLE 1

| | Stem | Bud length (mm) | | | | | Mean of | Mean of | Softness |
|---|---|---|---|---|---|---|---|---|---|
| Name of | tuber | | | | Average | Total | average | total | of stem |
| lineage | no. | Bud 1 | Bud 2 | Bud 3 | value | value | values | values | tuber |
| NT | 1 | 153.9 | 175.3 | | 164.6 | 329.2 | 160.7 | 321.4 | Soft |
| (Sassy) | 2 | 122.8 | 58.9 | | 90.9 | 181.7 | | | Soft |
| | 3 | 244.5 | 208.8 | | 226.7 | 453.3 | | | Soft |
| pSuehiro117 | 1 | 21.5 | 11.8 | | 16.7 | 33.3 | 19.5 | 31.1 | Slightly |
| #343 | | | | | | | | | soft |
| | 2 | 23.3 | | | 23.3 | 23.3 | | | Slightly |
| | | | | | | | | | soft |
| | 3 | 22.2 | 14.6 | | 18.4 | 36.8 | | | Slightly |
| | | | | | | | | | soft |
| pSuehiro117 | 1 | 33.9 | 25.5 | | 29.7 | 59.4 | 24.3 | 54.1 | Slightly |
| #389 | | | | | | | | | soft |
| | 2 | 14.5 | 18.9 | 16.2 | 16.5 | 49.6 | | | Slightly |
| | | | | | | | | | soft |
| | 3 | 26.8 | 26.4 | | 26.6 | 53.2 | | | Slightly |
| | | | | | | | | | soft |
| Stem tuber | | | | | | | | | Hard |
| stored at | | | | | | | | | |
| 4° C. | | | | | | | | | |

It was made clear from the results presented in FIGS. 6 to 8 and Table 1 that the genetically modified potato, where the SGA content was reduced, and budding was delayed, could be obtained by introducing deletion, insertion, or substitution into the CSLM gene of the potato.

Moreover, the hardness of the stem tubers of the genetically modified potato having the CSLM gene, into which deletion, insertion, or substitution was introduced, was increased, suggesting that aging was inhibited.

### Example 8: Propagation of Stem Tubers of Potato CSLM Genome-Edited Potato Lineage and Re-Examination of Suppression of Budding

The stem tubers of the potato (each of non-transformant (NT) of Sassy, transformant of pSuehiro117 #343, and transformant of pSuehiro117 #389) obtained in Example 7 were each grown in culture soil (PRO-MIX BX, produced by Premier Tech) inside a closed greenhouse (23° C., 16 hours of lighting (photon flux density: 32 μE/m2s)/8 hours of no lighting), to propagate stem tubers.

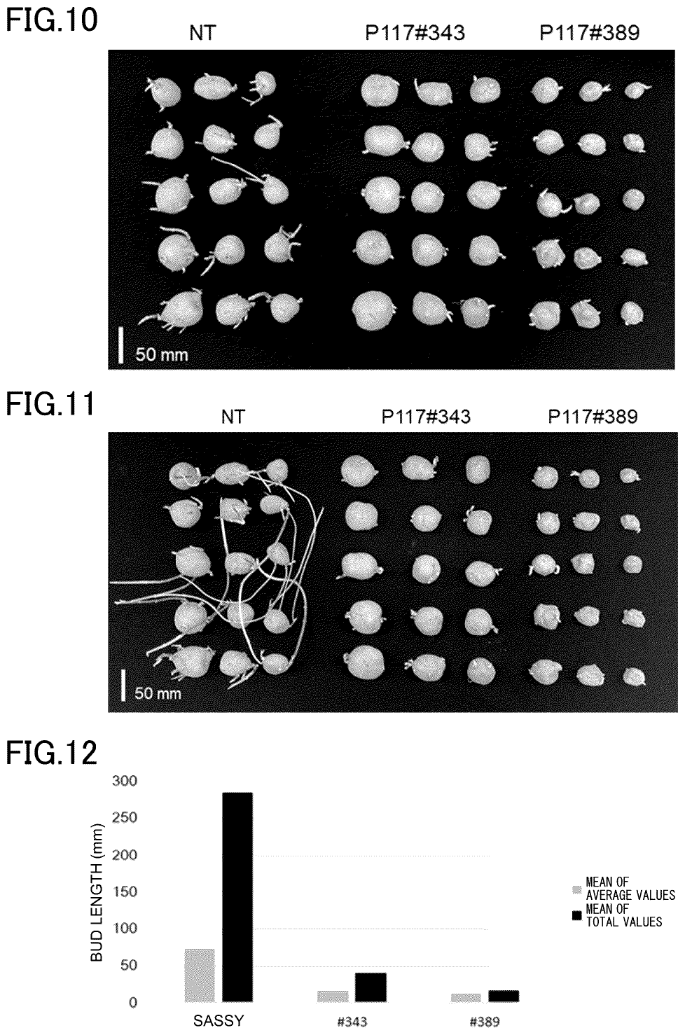

After harvesting, the stem tubers were stored for about 3 months at 5′C. After ending the dormant period, 15 stem tubers from each group were moved into a dark place at 20° C., and the budding test was started. FIG. 9 is a photograph depicting the state just after the start of the test; FIG. 10 is a photograph depicting the state of budding on the seventh week from the start of the test; and FIG. 11 is a photograph depicting the state of budding on the ninth week from the start of the test (left: non-transformant (NT) of Sassy, center: transformant of pSuehiro117 #343, right: transformant of pSuehiro117 #389). In the 9^{th} week from the start of the test, the length of each of the buds of 5 mm or longer was measured. The measured values, the average value of the bud lengths, and the total length of the bud lengths are presented in Table 2 and FIG. 12.

In Table 2, the stem tuber without buds having lengths of 5 mm or longer is denoted as “N.D.” and excluded from the calculation for the mean.

TABLE 2

| | | Bud length (mm) | | | | | | | | | | | Mean | Mean |
|---|---|---|---|---|---|---|---|---|---|---|---|---|---|---|
| Lineage | Tuber | | | | | | | | | | Ave. | Total | of | of |
| name | no. | Bud 1 | Bud 2 | Bud 3 | Bud 4 | Bud 5 | Bud 6 | Bud 7 | Bud 8 | | value | value | ave. | total |
| NT | 1 | 60 | 49 | 37 | 30 | 29 | 19 | 12 | 10 | | 31 | 246 | 73 | 284 |
| (Sassy) | 2 | 186 | 117 | 26 | 20 | 19 | 15 | 12 | 8 | 6 | 45 | 409 | | |
| | 3 | 185 | 181 | 52 | 24 | 16 | | | | | 92 | 458 | | |
| | 4 | 62 | 43 | 34 | 11 | | | | | | 38 | 150 | | |
| | 5 | 95 | 11 | | | | | | | | 53 | 106 | | |
| | 6 | 107 | 60 | 33 | 29 | 15 | | | | | 49 | 244 | | |
| | 7 | 177 | 25 | 13 | | | | | | | 72 | 215 | | |
| | 8 | 25 | 20 | 12 | | | | | | | 19 | 57 | | |
| | 9 | 174 | 74 | 25 | 22 | 21 | 19 | 18 | | | 50 | 353 | | |
| | 10 | 112 | 108 | 31 | | | | | | | 84 | 251 | | |
| | 11 | 193 | 17 | 16 | 16 | | | | | | 61 | 242 | | |
| | 12 | 295 | 190 | 168 | | | | | | | 218 | 653 | | |
| | 13 | 256 | 175 | 19 | 19 | | | | | | 117 | 469 | | |
| | 14 | 252 | 18 | | | | | | | | 135 | 270 | | |
| | 15 | 64 | 33 | 28 | 11 | | | | | | 34 | 136 | | |
| pSuehiro117 | 1 | 31 | 24 | 7 | | | | | | | 21 | 62 | 16 | 39 |
| #343 | 2 | 22 | 15 | 11 | 6 | | | | | | 14 | 54 | | |
| | 3 | 32 | 31 | 15 | | | | | | | 26 | 78 | | |
| | 4 | 12 | 8 | | | | | | | | 10 | 20 | | |
| | 5 | 9 | 7 | 7 | 7 | 6 | | | | | 7 | 36 | | |
| | 6 | 12 | 9 | | | | | | | | 11 | 21 | | |
| | 7 | 19 | 14 | 10 | | | | | | | 14 | 43 | | |
| | 8 | 34 | 19 | 13 | | | | | | | 22 | 66 | | |
| | 9 | 7 | | | | | | | | | 7 | 7 | | |
| | 10 | 30 | | | | | | | | | 30 | 30 | | |
| | 11 | 7 | 7 | 8 | | | | | | | 7 | 22 | | |
| | 12 | 16 | | | | | | | | | 16 | 16 | | |
| | 13 | 10 | | | | | | | | | 10 | 10 | | |
| | 14 | 46 | 45 | 19 | | | | | | | 37 | 110 | | |
| | 15 | 7 | 7 | | | | | | | | 7 | 14 | | |
| pSuehiro117 | 1 | 5 | | | | | | | | | 5 | 5 | 12 | 16 |
| #389 | 2 | 16 | 14 | | | | | | | | 15 | 30 | | |
| | 3 | 16 | 15 | 6 | | | | | | | 12 | 37 | | |
| | 4 | 10 | | | | | | | | | 10 | 10 | | |
| | 5 | 14 | | | | | | | | | 14 | 14 | | |
| | 6 | 13 | | | | | | | | | 13 | 13 | | |
| | 7 | N.D. | | | | | | | | | | | | |
| | 8 | 14 | | | | | | | | | 14 | 14 | | |
| | 9 | N.D. | | | | | | | | | | | | |
| | 10 | 14 | 11 | | | | | | | | 13 | 25 | | |
| | 12 | 7 | | | | | | | | | 7 | 7 | | |
| | 12 | 9 | | | | | | | | | 9 | 9 | | |
| | 13 | N.D. | | | | | | | | | | | | |
| | 14 | 14 | | | | | | | | | 14 | 14 | | |
| | 15 | 16 | | | | | | | | | 16 | 16 | | |

It was found from the results of FIGS. 9 to 12 and Table 2 that genetically modified potatoes whose budding were delayed were obtained by introducing deletion, insertion, or substitution into the CSLM gene of the potatoes.

For example, embodiments of the present invention include the followings and the like.

<1> A genetically modified potato, including:
a CSLM gene into which deletion, insertion, or substitution is introduced.

<2> The genetically modified potato according to <1>, wherein a glycoalkaloid content of the genetically modified potato is 50% or less, relative to a glycoalkaloid content of a genetically unmodified potato.

<3> The genetically modified potato according to <2>, wherein the glycoalkaloid is solanine or chaconine.

<4> The genetically modified potato according to any one of <1> to <3>,

wherein a bud length of the genetically modified potato in a dark place after an end of a post-harvest dormant period is 50% or less relative to a bud length of a genetically unmodified potato.

<5> A method for producing a genetically modified potato, the method including:
introducing deletion, insertion, or substitution into a CSLM gene of a potato.

<6> A genome editing method for a potato, the genome editing method including:
introducing deletion, insertion, or substitution into a CSLM gene of a potato using a genome editing agent.

<7> The genome editing method for the potato, according to <6>,

wherein the genome editing agent includes:
a protein binding to a CSLM gene, a nucleic acid encoding the protein binding to the CSLM gene, a guide RNA targeting a CSLM gene, or a nucleic acid encoding the guide RNA targeting the CSLM gene; and
an enzyme of nucleic acid metabolism or a nucleic acid encoding the enzyme of nucleic acid metabolism.

<8> The genome editing method for the potato, according to <7>,

wherein the enzyme of nucleic acid metabolism includes a nuclease or a deaminase.

<9> A composition for producing a genetically modified potato, the composition including:
a protein binding to a CSLM gene, a nucleic acid encoding the protein binding to the CSLM gene, a guide RNA targeting a CSLM gene, or a nucleic acid encoding the guide RNA targeting the CSLM gene; and
an enzyme of nucleic acid metabolism or a nucleic acid encoding the enzyme of nucleic acid metabolism.

<10> A method for determining a genetically modified potato, the method including:
determining whether a potato is a genetically modified potato or not based on the presence or absence of deletion, insertion, or substitution of a CSLM gene of the potato as an index.

1. A genetically modified potato, comprising:
a CSLM gene into which deletion, insertion, or substitution is introduced,
wherein a solanine content, or a chaconine content, or both the solanine content and the chaconine content of the genetically modified potato is 20% or less, relative to a solanine content, or a chaconine content, or both the solanine content and the chaconine content of a genetically unmodified potato, respectively.

2. (canceled)

3. (canceled)

4. The genetically modified potato according to claim 1,
wherein a bud length of the genetically modified potato in a dark place after an end of a post-harvest dormant period is 50% or less relative to a bud length of a genetically unmodified potato.

5. A method for producing a genetically modified potato, the method comprising:
introducing deletion, insertion, or substitution into a CSLM gene of a potato to produce a genetically modified potato,
wherein a solanine content, or a chaconine content, or both the solanine content and the chaconine content of the genetically modified potato is 20% or less, relative to a solanine content, or a chaconine content, or both the solanine content and the chaconine content of a genetically unmodified potato, respectively.

6. The method according to claim 5,
wherein the introducing deletion, insertion, or substitution into a CSLM gene of a potato is carried out by using a genome editing agent.

7. The method according to claim 6,
wherein the genome editing agent includes:
a protein binding to a CSLM gene, a nucleic acid encoding the protein binding to the CSLM gene, a guide RNA targeting a CSLM gene, or a nucleic acid encoding the guide RNA targeting the CSLM gene; and
an enzyme of nucleic acid metabolism or a nucleic acid encoding the enzyme of nucleic acid metabolism.

8. The method according to claim 7,
wherein the enzyme of nucleic acid metabolism includes a nuclease or a deaminase.

9. A composition for producing a genetically modified potato, the composition comprising:
a protein binding to a CSLM gene, a nucleic acid encoding the protein binding to the CSLM gene, a guide RNA targeting a CSLM gene, or a nucleic acid encoding the guide RNA targeting the CSLM gene; and
an enzyme of nucleic acid metabolism or a nucleic acid encoding the enzyme of nucleic acid metabolism,
wherein a solanine content, or a chaconine content, or both the solanine content and the chaconine content of the genetically modified potato is 20% or less, relative to a solanine content, or a chaconine content, or both the solanine content and the chaconine content of a genetically unmodified potato, respectively.

10. (canceled)


SEQUENCE LISTING (23 sequences)

SEQ ID NO: 1 - protein, 480 aa, Solanum tuberosum
MELNKSTVPQPITTIYRLHMFIHSIIMVALIYYRVSNLFKFENILSLQALAWVLITFGEFSFILKWFFGQGTRYRPVERDVFPENITCKDSDLPPIDVMVFTANPKKEPIVDVMNTVISAMALDYPTDKLAVYLADDGGCPLSLYAMEEACVFAKLWLPFCRKYGIKTRCPKAFFSPLGDDERVLKNDDFDAEMKEIKLKYEEFQQNVERAGESGKINGNVVPDRASFIKVINDRKAESEKSADDLTKMPLLVYVSRERRFNRLHHFKGGSANALLRVSGIMSNAPYILVLDCDFFCHDPISARKAMCFHLDPKLSSDLAYVQFPQVFYNVSKSDIYDVKIRQAYKTIWHGMDGIQGPVLSGTGYFLKRKALYTSPGVKEEYLSSPEKHFGRSKKFLASLEEKNGYVKAEKVISEDIVEEAKTLATCAYEDGTHWGQEANWLFIRLSFGEHFYWLSITLQRVEIDLFVSRQAIFLGLCPS

SEQ ID NO: 2 - DNA, 1443 bp, Solanum tuberosum
atggagctcaacaaaagcactgttccacaacctatcaccaccatataccgactccacatgtttatccactctataatcatggttgcattaatatactaccgtgtatctaatttgtttaaattcgaaaacattctgagtttacaagcacttgcttgggtactcatcacttttggtgaatttagtttcattctcaagtggttcttcggacaaggaactcgttatcgccctgttgaaagagatgttttccctgaaaacataacttgcaaagattccgatctaccaccaattgacgtaatggtattcactgccaatcctaagaaagagccaattgtggatgtcatgaacactgtgatatccgcaatggctcttgattatcctacggataaattggctgtgtatctggctgatgatggaggatgtcctttgtcattgtacgccatggaagaagcatgtgtgtttgcaaagctgtggctacctttctgtaggaagtatggaattaaaactagatgccctaaagcgtttttttctcctttaggagatgatgaacgtgttcttaagaatgatgattttgatgctgaaatgaaagaaattaaattgaaatatgaagagttccagcagaatgtggaacgtgctggtgaatctggaaaaatcaatggtaacgtagtgcctgatagagcctcgtttattaaggtaataaacgacagaaaagcggagagcgaaaagagtgccgatgatttaacgaaaatgcccttgctagtttatgtatcccgtgaaagaagattcaaccgtcttcatcacttcaagggtggatctgcaaatgctcttcttcgagtttctggaataatgagtaatgccccctatatactggtgttagattgtgatttcttctgtcatgatccaatatcagctaggaaggcaatgtgttttcatcttgatccaaagctatcatctgatttagcttatgttcagttccctcaagtcttttacaatgtcagcaagtccgatatttatgatgtcaaaattagacaggcttacaagacaatatggcatggaatggatggtatccaaggcccagtgttatcaggaactggttattttctgaagaggaaggcgttatacacgagtccaggagtaaaggaggagtatcttagttcaccggaaaagcattttggaaggagtaaaaagttccttgcttcactagaggagaaaaatggttatgttaaggcagagaaagtcatatcagaagatatcgtagaggaagctaagaccttagctacttgtgcatatgaggatggcacacattggggtcaagaggcaaattggttattcatacgattgtcatttggagagcacttttactggttatctattacactgcaaagggtggagatcgacttatttgtatccagacaggccatctttcttgggttgtgccccagttga

SEQ ID NO: 3 - DNA, 49 bp, Solanum tuberosum
tgcaaagattccgatctaccaccaattgacgtaatggtattcactgcca

SEQ ID NO: 4 - DNA, 17 bp, Solanum tuberosum
tgcaaagattccgatct

SEQ ID NO: 5 - DNA, 17 bp, Solanum tuberosum
aatggtattcactgcca

SEQ ID NO: 6 - DNA, 1716 bp, Solanum tuberosum
atgtcggatgctaaggtccccgcggccaccgttcaccaccctaggaggaagatccagttggtggactttcttctttcgttccgatggatcattgttatcttttttgtccttccattctcgttcttgtattacttctccatatatctaggggatgtgaaatctgagaagaaatcttacaagcaacgccagatggaacacgatgagaatgttaaagaggttgtgaagcgccttgaacagaggaatgcagcaaaggatggtcttgtctgcacggccaggcctccttgggttgttgttggaatgagaaacgtagactataaacgtgctcgtcattttgaagttgatctttctaagtttagaaatatacttgatattgacacggaacgtatggttgccaaagttgagcctcttgtcaatatgggccaaatgtcaagggtcaccatcccaatgaatctttcccttgcagttcttgctgagcttgatgatcttaccgttggtggtttgatcaatgggttcggggtcgaaggaagttctcacatctttggattgttctctgacactgttgtagcacttgaggttgttctagctgatggaaaggttgttagagctacaaaggacaacgaatattctgatcttttctacgctattccgtggtctcaagggacattggggcttcttgtttcagctgaaatcaagcttataccagttgatcaatacgtaaaacttacctacaaacctgtaaggggtaatcttaaagagcttgcacaggcttatgcggattcttttgcacctaaagatggagatcaggacaatccttctaaagttccagagatggtagaaggcatgatttatggtccaacggaaggtgttatgatgaccggtatgtatgcttcgaagaaagaagccaaacgaaggggtaatgtaatcaacaattatggttggtggttcaaaccatggttttaccaacacgctcaaactgcactgaaaagaggggagtttgtggagtacattccaactagggactactaccacaggcacacgagatcgttgtattgggaagggaaactaattcttccatttggtgatcagttctggtttaggttcctcttaggatggctcatgccaccaaagattgctctgctcaaagccacacaaagtgaggctattagaaactattaccatgaccatcatgtcattcaggatctgcttgttcctctttacaaggttggcgattgtctcgagtgggttcaccgcgagatggaggtatatcccatttggctctgcccacacagaatttacaagctgcctgtgagacctatgatctaccctgaaccagggttcgagaaacacaaaaggcagggtgacaccgaatatgcacaaatgtacactgatgttggtgtgtactatgttccgggagcagtcctgaggggtgagccatttgatggttcagagaaatgccgccaacttgagctttggttgatagaaaaccatggatttcaggctcaatacgcggtgactgagctgacagagaagaatttctggaggatgtttgataacagtctgtacgagcaatgcagaaggaagtacaaagctatcggaacgttcatgagtgtgtactataaatcgaagaaaggaaggaagacagagaaggaagtgcaggaagctgagcaagagaaagctgaacaagagactcctgaagctgatgaacctgcaaattga

SEQ ID NO: 7 - DNA, 29 bp, Artificial Sequence
  primer1
gagctctagaccctaggaggaagatccag

SEQ ID NO: 8 - DNA, 31 bp, Artificial Sequence
  primer2
ggatccatatgcgtttctcattccaacaaca

SEQ ID NO: 9 - DNA, 19 bp, Artificial Sequence
  primer3
taaagcacgaggaagcggt

SEQ ID NO: 10 - DNA, 19 bp, Artificial Sequence
  primer4
gcacaacagacaatcggct

SEQ ID NO: 11 - DNA, 20 bp, Artificial Sequence
  primer5
ctctgctcaaagccacacaa

SEQ ID NO: 12 - DNA, 20 bp, Artificial Sequence
  primer6
tcaattcgcaggttcatcag

SEQ ID NO: 13 - DNA, 1380 bp, Solanum tuberosum
atggccaagactactaatggatttaaggggcatgatatgttggcaccttttactgcggcatggatgattgatatgggacctttagttatagatagagcggagggttcttatgtctatgacgtaaatggaaagaagtaccttgattctttatctggtttatggtgcacagtattaggggggagtgagcctcgtcttattgaagctgcaaataaacaactcaataaattggcattttaccattcattttggaatcgttccacaaaaccatctttggatcttgcaaaggagctcataaatatgtttactgcaaataagatgggaaaagtttttttcacaaatagtggatcagaagctaatgacactcaggtgaagttggtgtggtattacaacaatgccattgggaggccaaacaaaaagaaaattatttctcgaaaaaatgcataccatggttccacttatatgactgccggtctctctgggcttccttcactacatctaaaatttgatttgccacctccatatattcttcacactgattgccctcattattggaacaatcacttgccaggtgagacagaagaggagtactcaactaggttggcaaataatttggaaaatcttatactcaaagagggtcctgaaacagttgctgctttcattgcggagccagtcatgggaggagcaggtgtcataattcctccggaaacctattttgagaagatccaagctgttttaaagaagtatgacattcttttcatcgcggatgaggtgatatgtggatttggaagacttgggacaatgtttggctgtgataagtacaacattaaacctgatcttgtctctatagcaaaggctctttctggtggatatataccaattggtgctgtgcttgtaagcgaagaaatttccaaggtcataagtgctcaaagcaatcaacttggtgttttttgccatggatttacttattctggacaccctgttgcgtgtgcggttgcattggaggcactaaagatctataaggaaaaaaatattactgaggtagtgaacaaattatcaccaaagtttcaagaaggtttgaaagcatttatcgacagtcccataattggggagataaggggaactggtttggtactttctaccgagtttgttgatagcaaatcttctaatgatccttttccactagaatggggtgttggtacatattttggatcacaatgtgagaagcatgggatgttggtaagtttttctggtgatcacgtaaatatggctcctccattcaccttgagtcttgaagaacttgatgagatgataagcatatatgggaaagcattgaaggatactgaaaaaagagtggaagaactcaagtctcagaaaaagtaa

SEQ ID NO: 14 - DNA, 31 bp, Artificial Sequence
  primer7
gagctctagatatttgatttgccacctccat

SEQ ID NO: 15 - DNA, 30 bp, Artificial Sequence
  primer8
ggatccatatgcttacaagcacagcaccaa

SEQ ID NO: 16 - DNA, 22 bp, Artificial Sequence
  primer9
tggggtgttggtacatattttg

SEQ ID NO: 17 - DNA, 20 bp, Artificial Sequence
  primer10
ttcctctttggctttctcca

SEQ ID NO: 18 - DNA, 25 bp, Artificial Sequence
  U1153
gtttaaattcgaaaacattctgagt

SEQ ID NO: 19 - DNA, 20 bp, Artificial Sequence
  U1154
atgacaaaggacatcctcca

SEQ ID NO: 20 - DNA, 19 bp, Artificial Sequence
  NP2
taaagcacgaggaagcggt

SEQ ID NO: 21 - DNA, 19 bp, Artificial Sequence
  NP3
gcacaacagacaatcggct

SEQ ID NO: 22 - protein, 715 aa, Solanum tuberosum
MKKTMELNKSTVPQPITTIYRLHMFIHSIIMVALIYYRVSNLFKFENILSLQALAWVLITFGEFSFILKWFFGQGTRYRPVERDVFPENITCKDSDLPPIDVMVFTANPKKEPIVDVMNTVISAMALDYPTDKLAVYLADDGGCPLSLYAMEEACVFAKLWLPFCRKYGIKTRCPKAFFSPLGDDERVLKNDDFDAEMKEIKLKYEEFQQNVERAGESGKINGNVVPDRASFIKVINDRKAESEKSADDLTKMPLLVYVSRERRFNRLHHFKGGSANALLRVSGIMSNAPYILVLDCDFFCHDPISARKAMCFHLDPKLSSDLAYVQFPQVFYNVSKSDIYDVKIRQAYKTIWHGMDGIQGPVLSGTGYFLKRKALYTSPGVKEEYLSSPEKHFGRSKKFLASLEEKNGYVKAEKVISEDIVEEAKTLATCAYEDGTHWGQEIGYSYDCHLESTFTGYLLHCKGWRSTYLYPDRPSFLGCAPVDMQGFSSQLIKWVAALTQAGLSHLNPITYGFSSRMKTLQCMCYAYLIYFTLYSWGMVLYASVPSIGLLFGFQVYPDVHDPWFAVYVIAFISAILENMSESIPDGGSFKSWWMEYRALMMMGVSAIWLGGLKAILDRIIGTEGEKLYLSDKAIDKEKLKKYEKGKFDFQGIGILAVPLIAFSLLNLVGFIVGANHVFITMNYAGVLGQLLVSSFFVFVVVTVVIDVVSFLKVS

SEQ ID NO: 23 - DNA, 2148 bp, Solanum tuberosum
atgaaaaaaaccatggagctcaacaaaagcactgttccacaacctatcaccaccatataccgactccacatgtttatccactctataatcatggttgcattaatatactaccgtgtatctaatttgtttaaattcgaaaacattctgagtttacaagcacttgcttgggtactcatcacttttggtgaatttagtttcattctcaagtggttcttcggacaaggaactcgttatcgccctgttgaaagagatgttttccctgaaaacataacttgcaaagattccgatctaccaccaattgacgtaatggtattcactgccaatcctaagaaagagccaattgtggatgtcatgaacactgtgatatccgcaatggctcttgattatcctacggataaattggctgtgtatctggctgatgatggaggatgtcctttgtcattgtacgccatggaagaagcatgtgtgtttgcaaagctgtggctacctttctgtaggaagtatggaattaaaactagatgccctaaagcgtttttttctcctttaggagatgatgaacgtgttcttaagaatgatgattttgatgctgaaatgaaagaaattaaattgaaatatgaagagttccagcagaatgtggaacgtgctggtgaatctggaaaaatcaatggtaacgtagtgcctgatagagcctcgtttattaaggtaataaacgacagaaaagcggagagcgaaaagagtgccgatgatttaacgaaaatgcccttgctagtttatgtatcccgtgaaagaagattcaaccgtcttcatcacttcaagggtggatctgcaaatgctcttcttcgagtttctggaataatgagtaatgccccctatatactggtgttagattgtgatttcttctgtcatgatccaatatcagctaggaaggcaatgtgttttcatcttgatccaaagctatcatctgatttagcttatgttcagttccctcaagtcttttacaatgtcagcaagtccgatatttatgatgtcaaaattagacaggcttacaagacaatatggcatggaatggatggtatccaaggcccagtgttatcaggaactggttattttctgaagaggaaggcgttatacacgagtccaggagtaaaggaggagtatcttagttcaccggaaaagcattttggaaggagtaaaaagttccttgcttcactagaggagaaaaatggttatgttaaggcagagaaagtcatatcagaagatatcgtagaggaagctaagaccttagctacttgtgcatatgaggatggcacacattggggtcaagagattggttattcatacgattgtcatttggagagcacttttactggttatctattacactgcaaagggtggagatcgacttatttgtatccagacaggccatctttcttgggttgtgccccagttgatatgcaaggtttctcctcacagctcataaaatgggttgctgcacttacacaagctggtttatcacatctcaatcccatcacttatggctttagtagcaggatgaaaactctccaatgcatgtgctatgcctatttgatatatttcactctttattcttggggaatggttctatatgctagtgttccttctattggccttttgtttggcttccaagtctatcccgatgtacatgatccatggtttgcagtgtatgtgattgctttcatatcggcaattttggagaatatgtcggagtcaattcctgatgggggatcatttaaatcttggtggatggaatacagggcactgatgatgatgggagttagtgcaatatggttaggaggattgaaagctatattagacaggataatcggaacagaaggagagaaattgtatttatcggacaaggcaattgacaaggaaaagctcaagaaatacgagaaggggaaatttgatttccaaggaatagggatacttgctgtaccattgatagcattttccttgttgaacctcgtaggcttcattgttggagctaatcatgtctttattactatgaactacgcaggtgtgcttggccaactcctcgtatcatccttcttcgtctttgtcgtggtcactgttgtcattgatgtcgtttctttcttaaaggtttcttaa